(19) United States
(12) Patent Application Publication
(10) Pub. No.: US 2008/0021257 A1
(43) Pub. Date: Jan. 24, 2008

(54) X-Ray Brachytherapy System and Device

(75) Inventors: Suranjan Roychowdhury, Plymouth, MN (US); Adam L. Gullickson, Richfield, MN (US); Kory P. Hamel, Bloomington, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 11/779,667

(22) Filed: Jul. 18, 2007

Related U.S. Application Data
(60) Provisional application No. 60/807,624, filed on Jul. 18, 2006.

(51) Int. Cl. A61N 5/00

(52) U.S. Cl. 600/3

(57) ABSTRACT

An improved x-ray brachytherapy system and related methods for delivering low dose radiation to a treatment location while minimizing damage to surrounding healthy tissue. Generally, the x-ray brachytherapy system can include a device controller, a device positioner, system imagery, and an x-ray brachytherapy probe having two or more delivery needles. The two or more delivery needles are operably coupled to at least one probe housing such that a tip spacing between the delivery needles is minimized when positioned at the treatment location. By reducing tip spacing at the treatment location, smaller radiation doses can be delivered so as to limit radiation exposure to surrounding healthy tissue.

### PRIORITY CLAIM

The present application claims priority to U.S. Provisional Application Ser. No. 60/807,624, filed Jul. 18, 2006 and entitled, “X-RAY BRACHYTHERAPY SYSTEM AND DEVICE”, which is herein incorporated by reference in its entirety.

### FIELD OF THE INVENTION

The present invention relates generally to radiation cancer treatments. More specifically, the present invention relates to an improved x-ray therapy treatment of cancerous tumors, and a device for administering the treatment.

### BACKGROUND OF THE INVENTION

The treatment of cancer can be accomplished in numerous ways, including surgery, radiation therapy, and chemotherapy. The treatment of prostate cancer, especially, requires a more focused application of radiation. Damage to the area surrounding the prostate gland, including the urethra, rectum, and the peripheral nerve bundle of the prostate gland, may occur if a radiation treatment encompasses too broad of a coverage area. For example, external beam radiation therapy can damage sensitive structures near a targeted area. Another form of radiation therapy, brachytherapy, limits unnecessary exposure to areas surrounding a targeted area. Brachytherapy is commonly used to treat prostate cancers, although it is also used to treat cancer in other parts of the body.

One type of brachytherapy used to treat prostate cancer involves the implantation of small radioactive metallic seeds into the prostate gland. The seeds are small, and many seeds (50-100) are implanted during the procedure. The radioactive seeds emit radiation into the prostate gland over a period of months, and typically the seeds are designed to remain radioactive for only a year or less. Care must be taken to avoid seed migration to surrounding healthy tissue. Once implanted, the seeds are intended to remain in the prostate gland permanently.

Another type of treatment of prostate cancer is high dose rate (HDR) brachytherapy. HDR brachytherapy is a temporary treatment, and involves placing many small catheters into the prostate gland, then introducing a series of radiation treatments into the prostate gland. The procedure is often computer controlled, allowing precise exposure times to radiation, and no radioactive material remains in the prostate gland after the treatment.

### SUMMARY OF THE INVENTION

The present invention comprises an improved x-ray brachytherapy system and device configured to more efficiently deliver radiation to a tumor location while minimizing the exposure to surrounding healthy tissue. Generally, a representative embodiment of an x-ray brachytherapy system of the disclosure comprises a device controller, a device positioner, system imagery, and a disposable x-ray probe having two or more delivery needles. Using the two or more delivery needles, the x-ray probe can direct radiation to targeted tissue within the body and serves as the interface between the x-ray system and a patient. The device controller controls radiation delivery at various dose rates and penetration depths in order to achieve desired efficacy rates. The device positioner ensures proper and predictable placement of the x-ray probe within a patient's body. The system imagery provides for accurate probe placement and visualization of a treatment region.

In one aspect of the present disclosure, an improved x-ray brachytherapy system provides for precise treatment of targeted tissue while avoiding permanent implantation of radioactive material within a patient. Single fraction treatment is possible with the present invention, greatly reducing inconvenience to a patient from repeated treatments. Generally, an x-ray brachytherapy probe comprises at least two low dose delivery needles, wherein the probe is configured to minimize spacing between tips on each delivery needles when positioned in the targeted tissue. By minimizing tip spacing, smaller doses of radiation can be delivered through each delivery needle such that damage to surrounding healthy tissue can be avoided.

In another aspect of the present invention, a x-ray brachytherapy prove can including at least one probe housing and a pair of delivery needles. In one embodiment, each delivery needle can be individually attached to a probe housing, such that by mounting the delivery needles in an off-set location or by making one needle longer than the other, a tip spacing at a target tissue area can be minimized. In another embodiment, the pair of delivery needles can be operably attached to a single probe housing.

In another aspect of the present invention, a known and predictable dose of radiation is delivered to the targeted tissue using two or more delivery needles. Tip spacing within targeted tissue can be minimized such the number of radiation doses and the size of the doses is reduced. As a result, radiation exposure to healthy tissue surrounding a targeted area is minimized.

In yet another aspect, the present invention does not require expensive facilities having bunker type radiation shielding. A low energy x-ray system according to the present disclosure offers increased safety to medical personnel when performing the procedure by reducing exposure to high radiation levels.

A still further aspect of the present invention is that a physician administering an x-ray treatment is able to manipulate various system parameters to customize the treatment to a particular individual.

Another aspect of the present invention comprises a method of providing an x-ray brachytherapy treatment, comprising generating an image of the targeted tissue with an imaging means, positioning a grid template assembly and a device positioner proximate the targeted tissue, securing an x-ray brachytherapy probe having multiple x-ray emitting needles on the device positioner, mapping a grid template location with a control system, inserting a needle portion of the x-ray brachytherapy probe through the mapped grid template location and into the patient to the targeted tissue, supplying a high voltage to the x-ray brachytherapy probe from the control system, and administering a radiation dose with the control system.

### BRIEF DESCRIPTION OF THE DRAWINGS

The invention may be more completely understood in consideration of the following detailed description of various embodiments of the invention in connection with the accompanying drawings, in which:

FIG. 1 is a cut-away perspective view of an x-ray brachytherapy probe of the prior art.

FIG. 2 is a perspective view of an embodiment of an x-ray brachytherapy device according to the present invention.

FIG. 3 is a perspective view of an embodiment of an x-ray brachytherapy device according to the present invention.

FIG. 4 is a side view of an embodiment of an x-ray brachytherapy device according to the present invention.

FIG. 5 is a perspective, partial cut-away view of an x-ray brachytherapy system for use with a patient according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

### DETAILED DESCRIPTION OF THE DRAWINGS

In the following detailed description of the present invention, numerous specific details are set forth in order to provide a thorough understanding of the present invention. However, it will be obvious to one skilled in the art that the present invention may be practiced without these specific details. In other instances, well-known methods, procedures, and components have not been described in detail so as to not unnecessarily obscure aspects of the present invention.

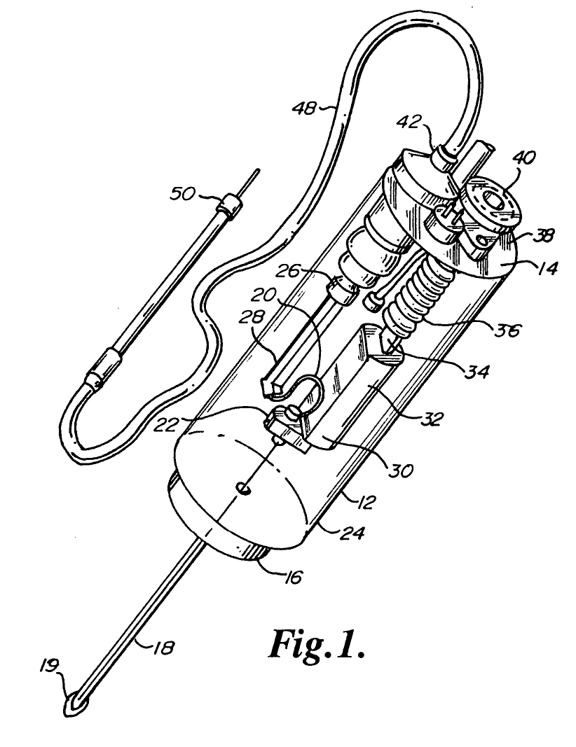

Referring to FIG. 1, there can be seen an example of a prior art x-ray brachytherapy (XRB) probe 10. XRB probe 10 generally comprises a housing 12 having a proximate end 14 and a distal end 16, and one or more needles 18 disposed on distal end 16. Needle 18 includes a tip portion 19 which may be adapted to pierce the skin of a patient by having a pointed, beveled, chiseled, or other shape suitable for piercing skin. Alternatively, tip portion 19 can have a rounded profile, and a trocar or similar piercing device is used in conjunction with XRB probe 10.

Within housing 12 is a cathode 20 to emit electrons into a vacuum and an anode 22 to collect the electrons. A vacuum (10⁻⁸ Torr) is maintained within housing 12. In a preferred embodiment, cathode 20 is diamond tipped, and anode 22 is aluminum with a gold coating. A quartz tube 24 having a high dielectric strength is provided to insulate the exterior of housing 12 when a high voltage, for example 10-150 kV, is applied. Also within housing 12 is a getter 26, a cathode holder 28 for securing cathode 20, an offset 30, an insulator 32, and an actuator shaft 34 coupled between insulator 32 and a bellows 36.

The proximate end 14 of housing 12 can include a back plane 38, a gap driver 40, and a pinch-off tube 42. A cable 48 is operably coupled to proximate end 14 of housing 12, such that cable 48 can transmit high voltage to XRB probe 10. A connector 50 is provided to couple cable 48 to a high voltage source. In a preferred embodiment, the high voltage source is between 20 kV and 50 kV.

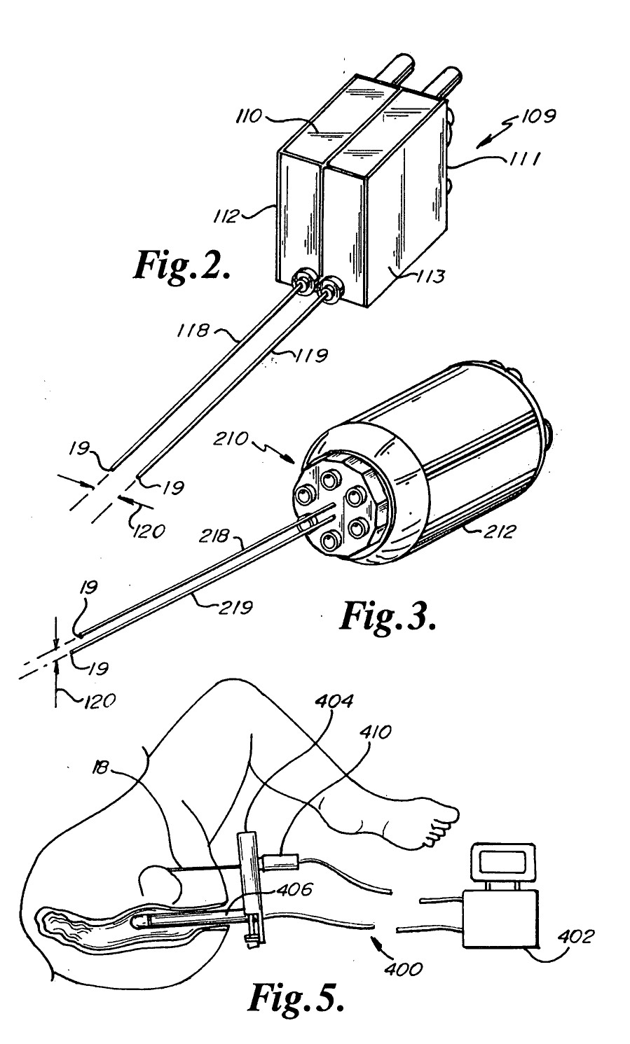

Referring now to FIGS. 2-4, various embodiments of XRB probes of the present invention are depicted. As illustrated in FIG. 2, an X-ray probe apparatus 109 can comprise two XRB probes 110 and 111, arranged proximate one another. Probe 110 and probe 111 each include a housing 112 and 113, respectively, and a needle 118 and 119, respectively. By altering the shape, or profile, of an XRB probe's housing, multiple XRB probes can be arranged together such that their needles are in close proximity to one another. It has been discovered that utilizing multiple needles during an XRB treatment increases the efficiency of the treatment and decreases the time needed to conduct the treatment. By using multiple needles, each emitting smaller amounts of radiation as opposed to a single needle emitting higher power radiation, the risk of damaging healthy tissue surrounding a targeted area is minimized. It is desirable to maintain multiple needles as close to one another as possible, as a targeted area is often small in size.

In the embodiment depicted in FIG. 2, each XRB probe 110 and 111 is adapted to emit x-rays from their respective needles 118 and 119. Each XRB probe 110 and 111 contains the same functional components as XRB probe 10 depicted in FIG. 1, with the functional components being re-arranged in such a way as to be contained within the respective housings 112 and 113, while positioning needles 118 and 119 in close proximity to one another. As illustrated in FIG. 2, housings 112 and 113 have a cross-sectional profile minimizing the distance between needles 118 and 119 and correspondingly, minimizing a tip spacing 120 between tip portions 19.

A further embodiment of the present invention is depicted in FIG. 3. Generally, an XRB probe 210 can comprise a housing 212 and a pair of needles 218 and 219. The depiction of only two needles should not be considered limiting, as an embodiment of XRB probe 210 having more than two needles is contemplated herein and fully intended to be within the spirit and scope of the present invention. XRB probe 210 contains substantially the same functional components as XRB probe 10 depicted in FIG. 1, with one set of functional components for each needle. As discussed above, the arrangement of multiple needles in close proximity to one another so as to minimize tip spacing 120 at the targeted area is advantageous as it not only increases the efficacy of the x-ray brachytherapy treatment but also minimizes damage to healthy tissue surrounding the targeted area.

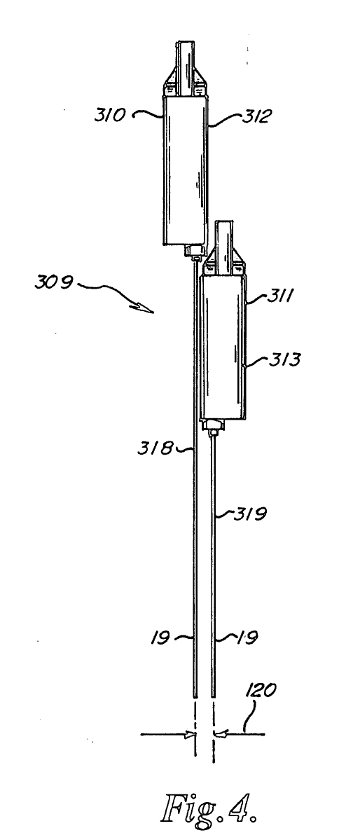

A still further embodiment of the present invention is depicted in FIG. 4. An XRB probe 310 and an XRB probe 311 are arranged generally proximate one another to form an X-ray probe apparatus 309. Probe 310 comprises a housing 312 and a needle 318, and probe 311 similarly comprises a housing 313 and a needle 318. Probe 310 and probe 311 are preferably arranged such that housings 312 and 313 are arranged in a staggered pattern, such that needles 318 and 319 are maintained in close proximity, wherein each needle has a unique length to facilitate the staggered arrangement of probes 310 and 311, as shown in FIG. 4. Utilizing the ability to stagger the XRB probes 310 and 311, tip spacing 120 is again minimized at the targeted treatment area. Each probe 310 and 311 contains substantially the same functional components as probe 10 depicted in FIG. 1. As discussed above, the arrangement of multiple needles in close proximity to one another is advantageous as it increases the efficiency of an x-ray brachytherapy treatment, as well as minimizes damage to healthy tissue surrounding the targeted area.

Referring now to FIG. 5, the present invention further comprises an improved x-ray brachytherapy system 400 configured to deliver radiation to a tumor location. System 400 comprises a device controller 402, a device positioner 404, system imagery 406, and an x-ray probe 410. Device controller 402 is adapted to control radiation delivery at various dose rates and penetration depths in order to achieve desired efficacy rates. Device positioner 404 is adapted to ensure proper and predictable placement of the x-ray probe within a patient's body. System imagery 406 is adapted to provide accurate probe placement and visualization of a treatment region. X-ray probe 410 is configured to direct radiation within the body and will serve as the interface between x-ray system 400 and a patient. In a preferred embodiment, x-ray probe 410 can comprise any of X-ray probe apparatus 109, X-ray probe 210 or X-ray probe apparatus 309. In some embodiments, X-ray brachytherapy system 400 can utilize a plurality of X-ray probes to achieve advantageous efficacy rates. For example, x-ray brachytherapy system 400 may utilize two or more of X-ray probe apparatus 109, X-ray probe 210 or X-ray probe apparatus 309 and combinations thereof.

The present invention further comprises a method of administering a radiation treatment to a patient. The patient is placed in the lithotomy position and administered either a spinal or general anesthesia. An imaging means is used to generate an image of the targeted area, wherein the imaging means may comprise computer axial tomography (CAT), magnetic resonance imaging (MRI), or in the case of treatment of a prostate gland, the preferred imaging means is transrectal ultrasound (TRUS). The TRUS probe is inserted into the patient's rectum to provide visualization of the prostate, and to aid with needle placement. A device positioner and a grid template are moved into position, and the grid template is positioned snugly against the patient's perineum.

An XRB probe having multiple x-ray emitting needles, such as the embodiments illustrated in FIGS. 2-4, is provided. The XRB probe can have a sharpened tip to break the skin of the patient, or a trocar may be used to create a pathway into the patient. The XRB probe is then fixed on the device positioner. Target areas (tumor regions) are pre-determined as the result of pre-treatment planning, and a control system maps the desired grid template hole to treat the target area. By using the TRUS probe for guidance, the XRB probe is inserted through the grid template into the perineum, and the needle tip portion of the XRB probe is guided to the tumor site. The control system supplies the high voltage source to the XRB probe, and the control system operates to the determined operating parameters. Once the treatment is performed, the XRB probe is removed and positioned at the next pre-determined treatment site. This process is repeated until all the treatment sites have been treated, at which point all of the equipment is removed away from the patient.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

1. An x-ray brachytherapy system for administering low dose treatment comprising:
a system controller,
a medical imaging system, and
at least two low dose delivery needles positioned such that a needle tip on each low dose delivery needle is insertable into a targeted treatment area.

2. The x-ray brachytherapy system of claim 1, wherein each low dose delivery needle is separately attached to a probe housing of an x-ray brachytherapy probe, each probe housing having a proximal connection end and a distal treatment end, wherein the low dose delivery needle is positioned on the distal treatment end.

3. The x-ray brachytherapy system of claim 2, wherein the low dose delivery needles are mounted to the probe housing in an off-center orientation such that the probe housings can be abuttedly positioned against each other such that the low dose delivery needles can be inserted so as to have minimal spacing between the needle tips.

4. The x-ray brachytherapy system of claim 2, wherein the probe housings are arranged in a staggered relation and wherein a first low dose delivery needle on a proximal housing is longer than a second low dose delivery needle on the distal housing such that the first low dose delivery needle and second low dose delivery needle can be inserted so as to have minimal spacing between the needle tips.

5. The x-ray brachytherapy system of claim 1, wherein the at least two low dose delivery needles are attached to a common probe housing.

6. The x-ray brachytherapy system of claim 1, wherein the needle tip on each low dose delivery needle comprises a sharpened tip for individually piercing the targeted tissue area.

7. The x-ray brachytherapy system of claim 1, wherein the medical imaging system is selected from the group consisting essentially of: computer axial tomography, magnetic resonance imaging and transrectal ultrasound.

8. A method for minimizing impact to healthy tissue surrounding a targeted treatment area using the x-ray brachytherapy system of claim 1.

9. A method of treating a targeted treatment area with low dose radiation comprising:
providing two or more delivery needles,
accessing a targeted treatment area through insertion of the two or more delivery needles into the targeted treatment area such that a tip spacing between the two or more delivery needles is reduced at the targeted treatment area, and
administering low dose radiation to the targeted treatment area through the two or more delivery needles.

10. The method of claim 9, further comprising:
forming an x-ray brachytherapy probe having a single probe housing such that the two or more delivery needles are operably coupled to the single probe housing.

11. The method of claim 9, further comprising:
forming two or more x-ray brachytherapy probes wherein each of the two or more delivery needles is individually attached to a probe housing; and
positioning the probe housings to minimize the tip spacing.

12. The method of claim 11, wherein each delivery needle is individually attached to the probe housing in an off-set orientation such that the probe housings can be abutted to minimize the tip spacing at the targeted treatment area.

13. The method of claim 11, wherein a first delivery needle on a first probe housing is longer than a second delivery needle on a second probe housing such that the first probe housing and second probe housing can be arranged in a staggered orientation to minimize the tip spacing at the targeted treatment area.

14. The method of claim 9, further comprising:
guiding the insertion of the two or more delivery needles with a medical imaging system arranged to visualize the targeted treatment area.

15. The method of claim 9, further comprising:
removing the two or more delivery needles from the targeted treatment area; and
repositioning the two or more delivery needles for reinsertion into a second targeted treatment area.

16. The method of claim 9, wherein the targeted treatment area comprises a prostate gland.

17. A low dose x-ray brachytherapy probe comprising:
at least one probe housing;
a first low dose delivery needle having a first tip; and
a second low dose delivery needle having a second tip,
wherein the probe housing is adapted such that the first tip and the second tip are insertable into a targeted treatment area such that first and second tip having a minimized tip spacing.

18. The low dose x-ray brachytherapy probe of claim 17, wherein the first low dose delivery needle is attached to a first probe housing and the second low dose delivery needle is attached to a second probe housing.

19. The low dose x-ray brachytherapy probe of claim 18, wherein both the first low dose delivery needle and the second low dose delivery needle are individually attached to the corresponding probe housing in an off-set orientation such that the first and second probe housings can be abuttedly positioned against each other such that the first and second low dose delivery needles can be inserted so as to have minimal spacing between the first and second tips.

20. The low dose x-ray brachytherapy probe of claim 18, wherein the first and second probe housings are arranged in a staggered relation and wherein the first low dose delivery needle is longer than the second low dose delivery needle such that the first and second low dose delivery needles can be inserted so as to have minimal spacing between the first and second tips.
